(19) United States
(12) Patent Application Publication
(10) Pub. No.: US 2022/0160274 A1
(43) Pub. Date: May 26, 2022

(54) NOISE ADJUSTMENTS TO CONTENT BASED ON COGNITIVE LOADS

(71) Applicant: Hewlett-Packard Development Company, L.P., Spring, TX (US)

(72) Inventors: Rafael Ballagas, Palo Alto, CA (US); Sarthak Ghosh, Palo Alto, CA (US)

(73) Assignee: Hewlett-Packard Development Company, L.P., Spring, TX (US)

(21) Appl. No.: 17/426,152

(22) PCT Filed: Apr. 19, 2019
(86) PCT No.: PCT/US2019/028271

§ 371 Date: Jul. 28, 2021

(51) Int. Cl. A61B 5/16; A61B 5/0533; A61B 5/0205; A61M 21/00

(52) U.S. Cl. CPC A61B 5/163; A61B 5/0533; A61M 2205/507; A61M 21/00; A61M 2021/005; A61B 5/02055

(57) ABSTRACT

In example implementations, an apparatus is provided. The apparatus includes a display, a biometric sensor, and a processor. The display is to play content. The biometric sensor is to collect biometric data of an individual. The processor is communicatively coupled to the display and the biometric sensor. The processor is to calculate a level of cognitive load based on the biometric data and to adjust an amount of noise in the content based on the level of cognitive load of the individual.

### BACKGROUND

Individuals can watch content for education, training, and the like. Different individuals may respond to the content in different ways. Some individuals may not remember what is in the content for different reasons.

### BRIEF DESCRIPTION OF THE DRAWINGS

FIG. 1 is a block diagram of an example system to adjust noise in content based on cognitive loads of the present disclosure;

FIG. 2 is a block diagram of an example virtual reality (VR) head mounted display to adjust noise in content based on cognitive loads of the present disclosure;

FIG. 3 is a block diagram of an examples of noise that can be adjusted in the content based on the cognitive loads of the present disclosure;

FIG. 4 is a flow chart of an example method for adjusting noise in content based on a cognitive load of the present disclosure;

FIG. 5 is a block diagram of an example non-transitory computer readable storage medium storing instructions executed by a processor to adjust noise in content based on a cognitive load of the present disclosure; and

FIG. 6 is an example graph of a relationship between cognitive load and performance.

### DETAILED DESCRIPTION

Examples described herein provide a method and system to adjust noise in content based on cognitive loads. As discussed above, some individuals may not remember what is in the content for different reasons. This may be detrimental to the individual as the content may be to provide information to the individual that is used for a job or the content may include information that may be on an exam.

Some individuals may not be engaged with the content. Some individuals may be overly stimulated by the content. In either case, the individual may not be at an “optimal” cognitive load and may have trouble remembering what is in the content.

A link has been found between performance and cognitive loads. An individual may better remember information shown in content when the individual has an “optimal” cognitive load. If the cognitive load of the individual is too low or too high, the individual may have trouble remembering information that is shown in the content.

Examples herein provide a method and system to adjust noise in the content to maintain an optimal cognitive load for an individual watching the content. For example, biometric data can be measured from the individual. The biometric data can be correlated to a level of cognitive load. The level of cognitive load can be compared against a threshold or a threshold range. If the level of cognitive load is within the threshold range, the content may be maintained. However, if the level of cognitive load is outside of the threshold range, the content can be adjusted to increase or decrease the amount of noise.

Adjusting the amount of noise in the content can help the individual to focus better on the content. The noise can be adjusted in either video content, audio content, or both video and audio content.

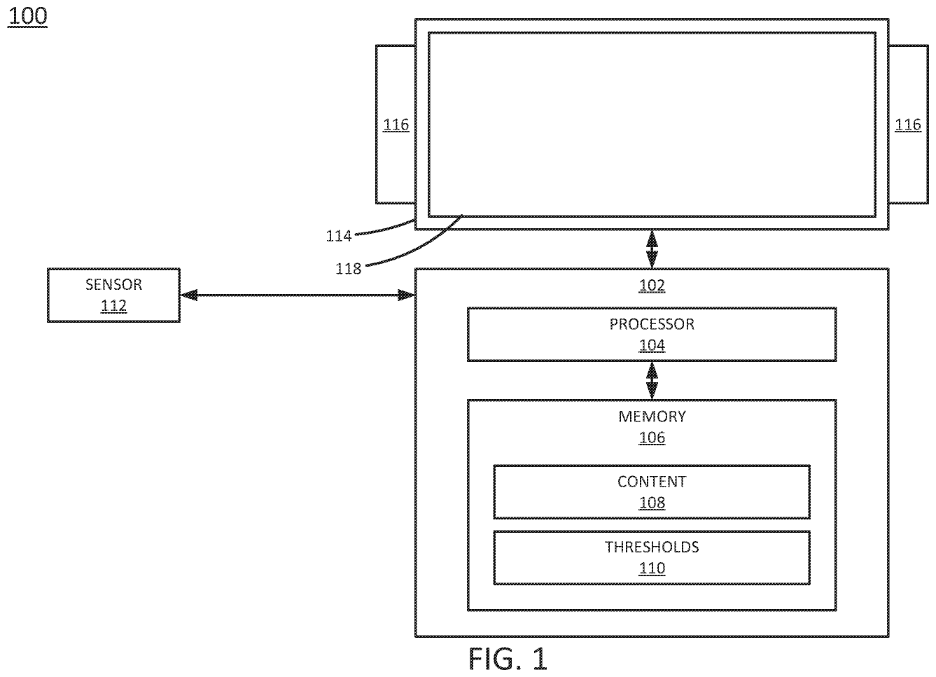

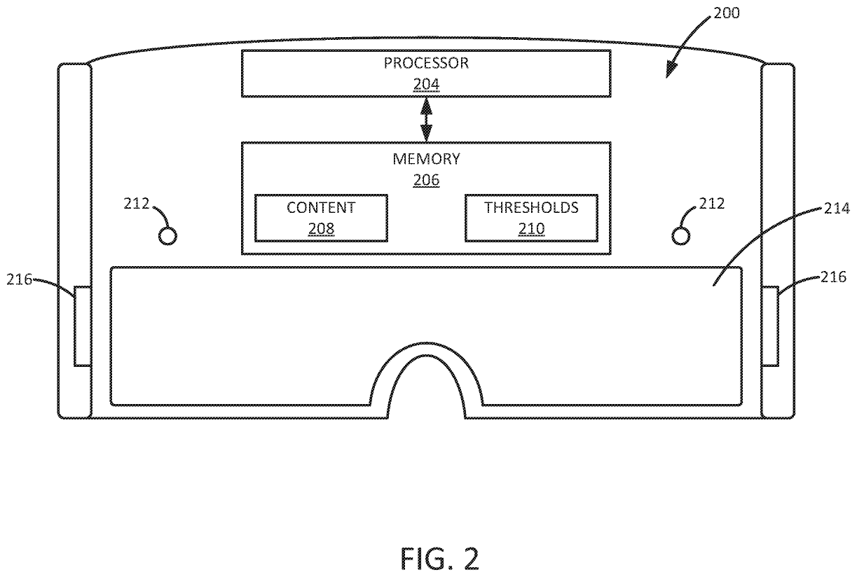

FIG. 1 illustrates an example system 100 of the present disclosure. The system 100 may be deployed as various separate components as a desktop computer, as a laptop computer, as a tablet computer, a virtual reality (VR) head mounted display (as illustrated in FIG. 2 and discussed in further details below), and the like.

In one example, the system 100 may include a controller 102, a sensor 112, and a content playing device 114. The controller 102 may be communicatively coupled to the sensor 112 and the content playing device 114.

The sensor 112 may be a biometric sensor. Although a single sensor 112 is illustrated in FIG. 1, it should be noted that any number of sensors 112 may be deployed. Different sensors 112 may be deployed to measure different types of biometric data or parameters. For example, the biometric data may include tracking pupil dilation from an eye tracking sensor, galvanic skin response (GSR) from a GSR sensor, heart rate from a heart rate monitor/sensor, body temperature from a body temperature sensor, and the like.

The sensor 112 may be located anywhere on an individual's body. For example, the sensor 112 may be worn on a hand, be worn as a helmet and located in front of a user's eyes, worn on the body or torso, or any combination thereof.

The biometric data measured by the sensor 112 may be transmitted to the controller 102. The controller 102 may process and/or analyze the biometric data to adjust an amount of noise in content that is displayed on the content playing device 114, as discussed in further details below.

In one example, the content playing device 114 may include a display 118 to provide visual content (e.g., videos, images, and the like) and speakers 116 to provide audio content. The display 118 may be monitor, a heads up display, a television, a laptop screen, and the like. The display 118 may be any type of device that can show images to an individual. The content may include video without audio, audio without video, or video and audio.

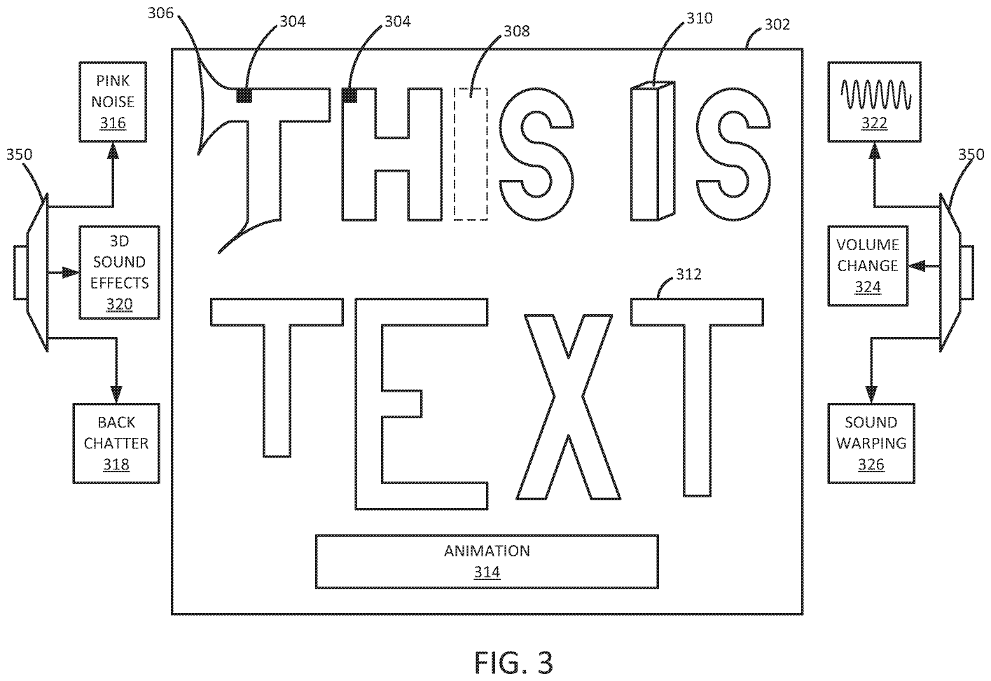

As discussed in further details below, “noise” may be visual noise or audible noise. Thus, adjustments to the amount of noise may include adding visual noise, adding audible noise, or a combination of both. “Noise” may be defined as any effect that hinders visual information transfer or audible/oral transfer of information. Examples of noise are illustrated in FIG. 3 and discussed in further details below.

In one example, the controller may include a processor 104 and a memory 106. The processor 104 may be communicatively coupled to the memory 106. The memory 106 may be any type of non-transitory computer readable medium. For example, the memory 106 may be a hard disk drive, a random access memory (RAM), a read only memory (ROM), and the like.

In one example, the memory 106 may include content 108 and thresholds 110. The content 108 may include multimedia (e.g., video, images, audio, and the like) that can be outputted by the content playing device 114. The content 108 may include educational content. For example, the content 108 may be a training video, an educational video, a lecture, and the like. The content 108 may be played for individuals so they can learn from the information that is conveyed in the content 108.

However, as noted above, there has been found to be a link between performance (e.g., the ability memorize, retain, or recall information from the content 108) and cognitive load. Cognitive load may be defined as a total amount of mental effort being used in the working memory. The amount or level of cognitive load may be associated with various biometric data that is measured from an individual. The level of cognitive load may be normalized to a value or score based on an estimated level of the cognitive load that is correlated to the biometric data that is measured.

For example, the biometric data may include pupil dilation, GSR, heart rate, body temperature and the like. A scale may be assigned to each item of biometric data. For example, different ranges of pupil dilation per unit of time may be assigned different scores between 1-5, 1 being indicative of a low cognitive load and 5 being indicative of a high cognitive load. Similarly, different ranges of GSR, heart rate, and body temperature may also be assigned a score between 1-5. The scores may be added, with or without weighting, to calculate an amount or level of cognitive load. It should be noted that although a scale of 1 to 5 is used as an example, any scoring scale or values can be used.

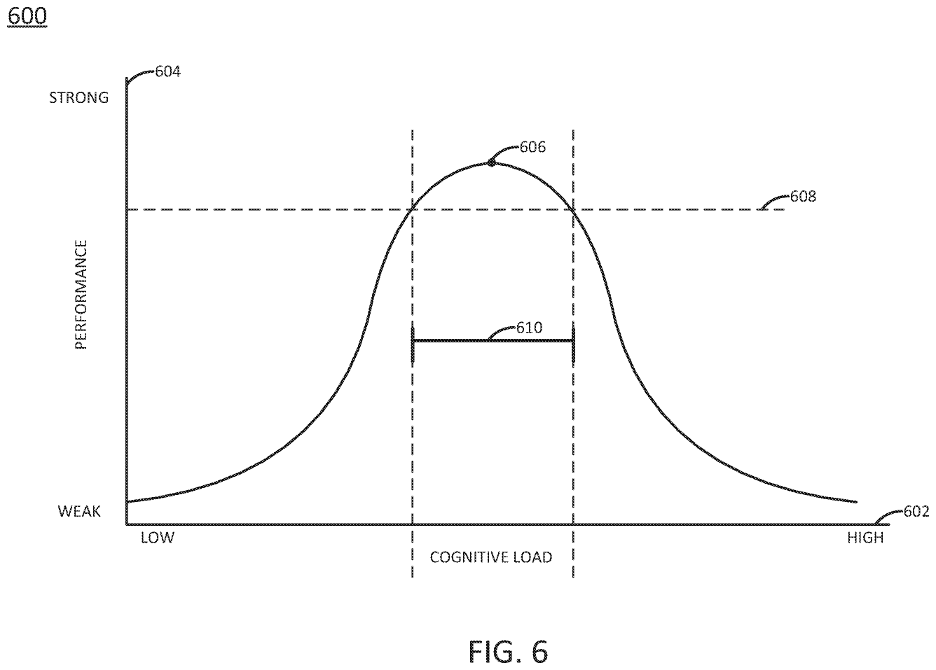

FIG. 6 illustrates an example of a graph 600 that shows a relationship between cognitive load 602 and performance 604. As noted above, the performance 604 may be a measure of how much information can be memorized, recalled, remembered, and the like from the content 108 that is watched by an individual.

As can be seen in the graph 600, a maximum level of performance 606 is achieved when the cognitive load 602 is not too high and not too low (e.g., at a midpoint between a lowest cognitive load and a highest cognitive load). As the cognitive load 602 increases, the performance 604 decreases. As the cognitive load 602 decreases, the performance 604 decreases. Thus, to maintain a desired level of performance 608, the cognitive load 602 may be maintained within a threshold or a threshold range 610. In one example, the threshold range 610 may be determined as a desired percentage within the maximum level of performance 606 (e.g., +/−5% of the maximum level of performance 606, +/−10% of the maximum level of performance 606, and the like). The threshold range 610 may be stored in the thresholds 110 in the memory 106 of the controller 102.

The threshold range 610 may be different for different individuals. In one example, an initialization process may be executed for an individual to determine the optimal cognitive load to obtain the maximum performance 606. The threshold range 610 can be determined for the particular individual.

Referring back to FIG. 1, the biometric data may be measured by the sensor 112, as noted above. The biometric data may be transmitted to the controller 102 via a wired or wireless connection and analyzed by the processor 104. The processor 104 may calculate the cognitive load of the individual based on the biometric data.

The cognitive load that is calculated may be compared to a threshold 110 to determine if the individual is at an optimal cognitive load for maximizing performance. If the cognitive load of the individual is outside of the threshold 110 (e.g., outside of the threshold range 610), the processor 104 may introduce noise into the content 108 that is played by the display 118 and the speakers 116. Adding noise may be counterintuitive, but adding noise may help the individual to focus harder on the information being conveyed by the content 108 and improve performance.

If the cognitive load is within the threshold 110, the processor 104 may leave the content 108 as is or remove the noise from the content 108. For example, the individual may have the proper amount of focus to maintain a high level of performance. Thus, no noise is added or the previously added noise may be removed.

The biometric data can be continuously measured by the sensor 112 and analyzed by the processor 104. Thus, the processor 104 may adjust an amount of noise in the content 108 in real-time as the individual is watching or consuming the content 108. Thus, the cognitive load of the individual may be maintained within a desired range as the individual is watching the content 108 to ensure that the individual is performing at an optimal level.

FIG. 2 illustrates an example VR head mounted display (HMD) 200 of the present disclosure. As noted above, the system 100 may be deployed as part of the VR HMD 200. The VR HMD 200 may be worn by an individual to consume the content.

In one example, the VR HMD 200 may include a processor 204, a memory 206, a sensor 212, a display 214, and speakers 216. The processor 204 may be communicatively coupled to the memory 206, the sensors 212, the display 214, and the speakers 216.

In one example, the sensors 212 may include video cameras that can track pupil dilation of the eyes of an individual wearing the VR HMD 200. In one example, VR HMD 200 may include other types of sensors 212 that may contact the skin on the forehead or temples of the individual's head. For example, the other types of sensors 212 may measure GSR, heart rate, body temperature, and the like.

The memory 206 may be any type of non-transitory computer readable medium. For example, the memory 206 may be a hard disk drive, a random access memory (RAM), a read only memory (ROM), and the like. The memory 206 may include content 208 and thresholds 210. The content 208 may be educational multimedia (e.g., video, images, audio, and the like) that is shown on the display 214 and heard through the speakers 216, similar to the content 108. In one example, the thresholds 210 may be calculated and stored similar to the way the thresholds 110 are calculated and stored, as discussed above with respect to FIG. 1.

The processor 204 may receive the biometric data measured by the sensor 212 and calculate a cognitive load based on the biometric data that is measured. The processor 204 may compare the cognitive load that is calculated against the threshold 210 to determine if the cognitive load is within the desired range.

If the cognitive load that is calculated is outside of the threshold 210 or desired range, then the processor 204 may add noise or adjust an amount of noise in the content 208 that is playing. If the cognitive load that is calculated is within the threshold 210 or desired range, then the processor 204 may leave the content 208 as is without adding noise. The processor 204 may remove noise that was previously added if the cognitive load that is calculated is within the threshold 210 or desired range.

FIG. 3 illustrates a display 302 and speakers 350 with examples of noise that can be added to the content 108 or 208. The display 302 and the speakers 350 may be similar to the display 118 and the speakers 116 illustrated in FIG. 1.

As noted above, the noise can be visual noise or audible noise. In one example, visual noise may include warping of edges 306, intentionally removed pixels 304, missing portions of the image 308 (e.g., missing text or graphics), three-dimensional effects 310, disproportionate stretching 312, animations 314, and the like. In one example, the audible noise may include pink or white noise 316, back chatter 318 (e.g., adding background conversations), three-dimensional sound effects 320 (e.g., adding sound from different perceptive angles via the speakers 350), modifications to the audio 322 (e.g., changing pitch, adding modulation, and the like), volume changes 324 (e.g., continuously or periodically changing the volume level as the audio is playing), sound warping 326, and the like.

In one example, the visual noise may be added without the audible noise. In one example, the audible noise may be added without the visual noise. In one example, the visual noise and the audible noise may both be added.

In one example, an initialization process may be executed for an individual. The initialization process may also test how the different types of noise affect the cognitive load and performance of an individual. For example, some individuals may respond better to missing pixels 304 or missing letters 308, while other individuals may respond better to three dimensional effects 310 or animations 314. Some individuals may respond better to pink noise 316, while other individuals may respond better to volume change 324.

In some examples, an individual may have different levels of response to the different types of noise. For example, an individual may have a bigger increase to cognitive load when seeing the warped edges 306 and a smaller increase to cognitive load when seeing disproportionate stretching 312. Thus, different types of noise may be added or removed to fine tune the cognitive load of the individual to be within the threshold 110.

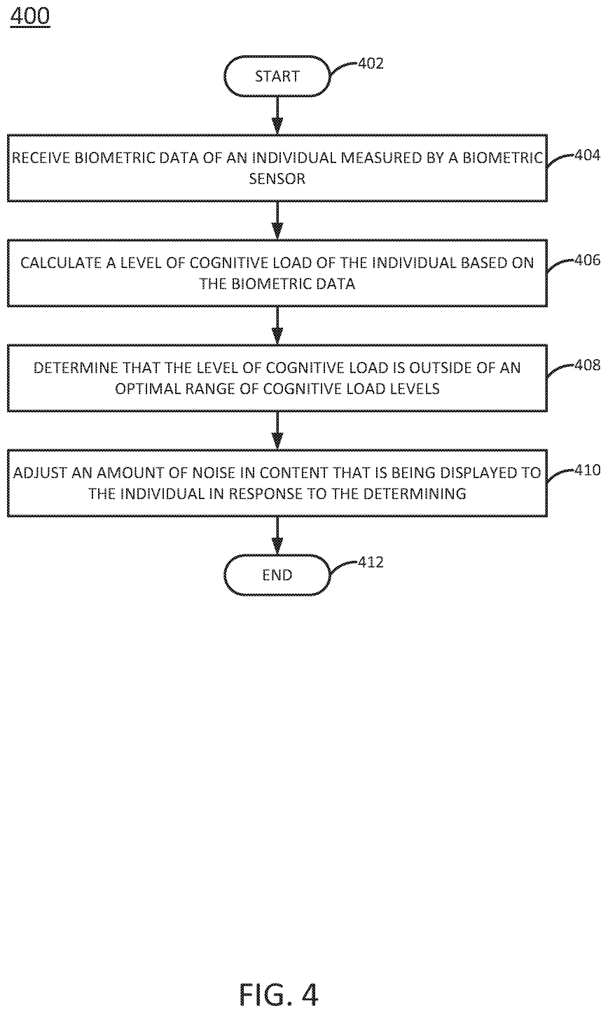

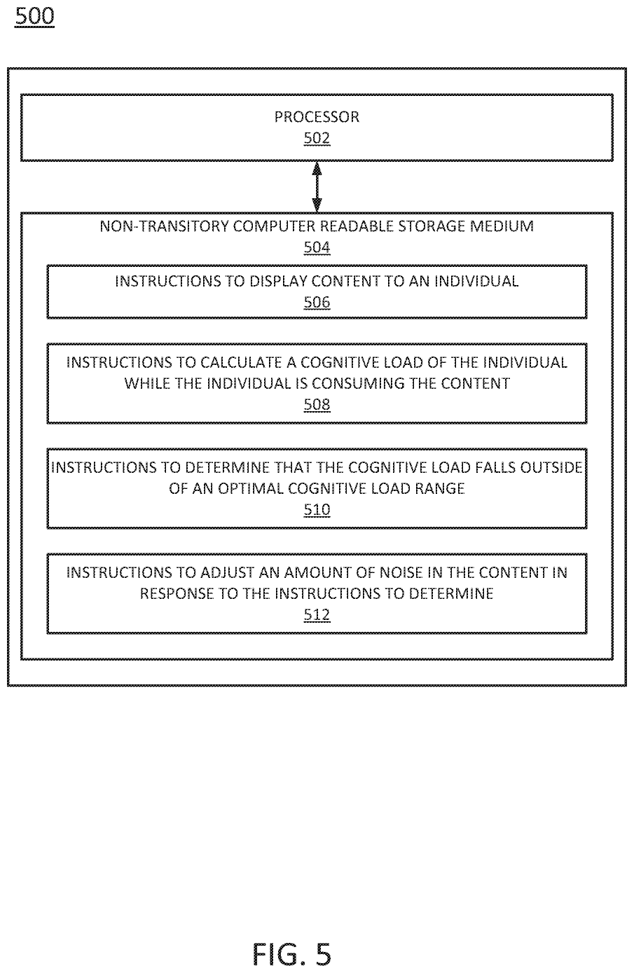

FIG. 4 illustrates a detailed flow chart of a method for adjusting noise in content based on a cognitive load of the present disclosure. In an example, the method 400 may be performed by the system 100 illustrated in FIG. 1, the VR head mounted display 200 illustrated in FIG. 2, or the apparatus 500 illustrated in FIG. 5, and described below.

At block 402, the method 400 begins. At block 404, the method 400 receives biometric data of an individual measured by a biometric sensor. The biometric sensors may include different sensors to measure different biometric data. In one example, the biometric sensor may include an eye tracking device to track pupil dilation. In another example, the biometric sensors may include sensors to measure GSR, heart rate, body temperature, and the like.

At block 406, the method 400 calculates a level of cognitive load of the individual based on the biometric data. In one example, the biometric data may be correlated to a level of cognitive load, as described above.

At block 408, the method 400 determines that the level of cognitive load is outside of an optimal range of cognitive load levels. The optimal range may be determined for an individual, as described above. For example, an initialization process may be used to determine the optimal range of cognitive load levels for the individual, what types of noise the individual responds to, and the like.

The level of cognitive load that is calculated based on the biometric data may be compared to the optimal range of cognitive load levels. If the level of cognitive load is outside of the optimal range of cognitive load levels, adjustments to the amount of noise in the content can be made. If the level of cognitive load is within the optimal range, then the content may continue to play without adjustments.

At block 410, the method 400 adjusts an amount of noise in content that is being displayed to the individual in response to the determining. In one example, if the calculated level of cognitive load of the individual is below the optimal range, then the amount of noise may be increased. For example, visual noise and/or audible noise may be added to the content.

If the calculated level of cognitive load of the individual is above the optimal range, then the amount of noise may be decreased. For example, visual noise and/or audible noise may be removed from the content. In one example, the type of noise that is added or removed to adjust the level of cognitive load of the individual to be within the optimal range may be based on results of the initialization process. As noted above, different individuals may respond differently to different types of noise. In addition, the cognitive load of the individual may change by different amounts based on the different types of noise.

In one example, the method 400 may be repeated continuously for the duration of the content. For example, if the content is a training video, the method 400 may be repeated continuously to adjust the amount of noise in the content in real-time. As a result, the individual consuming the content may be kept within the optimal range of cognitive loads to ensure that the individual can remember or recall the information provided by the content. At block 412, the method 400 ends.

FIG. 5 illustrates an example of an apparatus 500. In an example, the apparatus 500 may be the system 100 or the VR head mounted display 200. In an example, the apparatus 500 may include a processor 502 and a non-transitory computer readable storage medium 504. The non-transitory computer readable storage medium 504 may include instructions 506, 508, 510, and 512 that, when executed by the processor 502, cause the processor 502 to perform various functions.

In an example, the instructions 506 may include instructions to display content to an individual. The instructions 508 may include instructions to calculate a cognitive load of the individual while the individual is consuming the content. The instructions 510 may include instructions to determine that the cognitive load falls outside of an optimal cognitive load range. The instructions 512 may include instructions to adjust an amount of noise in the content in response to the instructions to determine.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

1. An apparatus, comprising:
a content playing device to output content;
a biometric sensor to collect biometric data of an individual;
a processor communicatively coupled to the content playing device and the biometric sensor, wherein the processor is to calculate a level of cognitive load based on the biometric data and to adjust an amount of noise in the content based on the level of cognitive load of the individual.

2. The apparatus of claim 1, further comprising:
a memory to store a threshold range for levels of cognitive loads that provide an optimal cognitive load.

3. The apparatus of claim 2, wherein the processor is to increase the amount of noise in the content when the level of cognitive load is below the threshold range for levels of cognitive loads.

4. The apparatus of claim 2, wherein the processor is to decrease the amount of noise in the content when the level of cognitive load is above the threshold range for levels of cognitive loads.

5. The apparatus of claim 1, wherein the biometric sensor comprises at least one of: a heart rate sensor, an eye-tracking sensor, a galvanic skin response (GSR) sensor, or a body temperature sensor.

6. The apparatus of claim 1, wherein the content playing device comprises a display and a speaker to play multimedia content.

7. The apparatus of claim 1, wherein the content playing device, the biometric sensor, and the processor are enclosed in a virtual reality head mounted display.

8. A method, comprising:
receiving, by a processor, biometric data of an individual measured by a biometric sensor;
calculating, by the processor, a level of cognitive load of the individual based on the biometric data;
determining, by the processor, that the level of cognitive load is outside of an optimal range of cognitive load levels; and
adjusting, by the processor, an amount of noise in content that is being displayed to the individual in response to the determining.

9. The method of claim 8, wherein the adjusting comprises:
increasing, by the processor, the amount of noise in the content when the level of cognitive load is below the optimal range of cognitive load levels.

10. The method of claim 8, wherein the adjusting comprises:
decreasing, by the processor, the amount of noise in the content when the level of cognitive load is above the optimal range of cognitive load levels.

11. The method of claim 8, wherein the amount of noise is adjusted in the video or the audio of the content.

12. The method of claim 8, wherein noise in the video of the content comprises at least one of: an introduction of blank pixels, a warping of edges, a disproportionate stretching of the content, an introduction of three-dimensional effects, blur, or added movement.

13. The method of claim 8, wherein noise in the audio of the content comprises at least one of: an introduction of pink noise, back-chatter, modulation, change in volume, change in pitch, three-dimensional audio effects, or sound warping.

14. A non-transitory computer readable storage medium encoded with instructions executable by a processor, the non-transitory computer-readable storage medium comprising:
instructions to display content to an individual;
instructions to calculate a cognitive load of the individual while the individual is consuming the content;
instructions to determine that the cognitive load falls outside of an optimal cognitive load range; and
instructions to adjust an amount of noise in the content in response to the instructions to determine.

15. The non-transitory computer readable storage medium of claim 14, wherein the cognitive load is calculated based on monitoring pupil dilation with an eye-tracking system.
